(19) United States
(12) Patent Application Publication
(10) Pub. No.: US 2018/0256867 A1
(43) Pub. Date: Sep. 13, 2018

(54) Nasal Delivery Device and Methods of Use

(71) Applicant: Bruce H. Levin, Oceanside, NY (US)

(72) Inventor: Bruce H. Levin, Oceanside, NY (US)

(21) Appl. No.: 15/915,046

(22) Filed: Mar. 7, 2018

Related U.S. Application Data
(60) Provisional application No. 62/468,120, filed on Mar. 7, 2017.

(51) Int. Cl. A61M 31/00; A61M 11/00; A61M 11/02

(52) U.S. Cl. CPC A61M 31/00; A61M 11/007; A61M 2210/0618; A61M 11/02; A61M 11/008

(57) ABSTRACT

A drug delivery system includes flexible and interchangeable nozzle and tip. In one embodiment, the present invention relates to a medical device for intranasal delivery of a medicament. The present invention effectively delivers an appropriate amount of medicament to the designated surface area. In one other embodiment., the present invention ensures that a complete dosage of the medicament is delivered, especially to specific areas in the nasal cavity such as the rear of the nasal cavity where the SPG is located.

### RELATED APPLICATIONS

This application claims priority to U.S. Provisional Patent Application Ser. No. 62/468,120, filed Mar. 7, 2017, titled the same and incorporated herein as if set out in full.

### STATEMENT REGARDING FEDERALLY SPONSORED RESEARCH & DEVELOPMENT

Not applicable.

### INCORPORATION BY REFERENCE OF MATERIAL SUBMITTED ON A COMPACT DISC

Not applicable.

### BACKGROUND OF THE INVENTION

Traditional devices for delivering drugs to the nasal cavity include syringed nose drops, pump spray devices, swabs, and propellant metered dose inhalers (MDI). Products such as SphenoCath® devices and Allevio Catheter have been in the market to help patients with their symptoms. Yet, these traditional devices have not been able to achieve the secure and efficient delivery of a medicament to maximize efficacy while helping mitigate undesired pulmonary absorption. For example, both eye dropper type devices and simple spray devices typically present medicament into the nasal cavity in a stream. The result is that much of the medicament simply runs out of the patient's nose or down the throat, and only a small amount of the drug is absorbed, with even less of the drug reaching the desired area in the nasal cavity.

Thus, there is a need for improved drug delivery systems to administer a desired dosage to the nasal epithelia or a predetermined area such as the sphenopalatine ganglion (hereinafter, the “SPG”).

### BRIEF SUMMARY OF THE INVENTION

In one embodiment, the present invention relates to a medical device for intranasal delivery of a medicament. The medicament may be any type of medicament suitable for nasal administration and delivery.

In one embodiment, the present invention relates to a medical device for delivery of a medicament to difficult to reach areas of a human body.

In other embodiments, the present invention ensures that a complete dosage of the medicament is delivered, especially to specific areas in the nasal cavity such as the rear of the nasal cavity where the SPG is located.

In other embodiments, the present invention ensures that a complete dosage of the medicament is delivered, especially to designated areas.

### BRIEF DESCRIPTION OF THE SEVERAL VIEWS OF THE DRAWINGS

In the drawings, which are not necessarily drawn to scale, like numerals may describe substantially similar components throughout the several views. Like numerals having different letter suffixes may represent different instances of substantially similar components. The drawings illustrate generally, by way of example, but not by way of limitation, a detailed description of certain embodiments discussed in the present document.

FIG. 1A is a perspective view of one embodiment of the present invention.

FIG. 1B is a perspective view of the embodiment of the present invention shown in FIG. 1A with a fitting that may be movable or fixed.

FIG. 2 is a perspective view of another embodiment of the present invention.

FIG. 3 is a perspective view of another embodiment of the present invention.

FIG. 4 is a perspective view of another embodiment of the present invention.

FIG. 5A is a perspective view of another embodiment of the present invention.

FIG. 5B is an exploded view of a dispenser that may be used with the present invention.

FIG. 5C is an exploded view of another dispenser that may be used with the present invention.

FIG. 6A is a perspective view of another embodiment of the present invention.

FIG. 6B is an exploded view of a dispenser that may be used with the present invention.

FIG. 7A is a perspective view of another embodiment of the present invention.

FIG. 7B is an exploded view of a dispenser that may be used with the present invention.

FIG. 8 is a perspective view of another embodiment of the present invention.

FIG. 9 is a perspective view of another embodiment of the present invention.

FIG. 10 is a perspective view of another embodiment of the present invention.

FIG. 11 is a perspective view of another embodiment of the present invention.

FIG. 12 is a perspective view of another embodiment of the present invention.

FIG. 13A is a perspective view of another embodiment of the present invention.

FIG. 13B is an exploded view of a dispenser that may be used with the present invention.

FIGS. 14, 15, 16, 17, 18, 19, 20 and 21 illustrate a method of deploying an embodiment of the present invention.

FIGS. 22-23 depict how the tip may be made from nonwoven fabrics.

FIGS. 24, 25, 26 and 27 depict weavings that may be used to form tips.

FIG. 28A is a perspective view of another embodiment of the present invention.

FIG. 28B is an exploded view of a dispenser that may be used with the present invention.

FIG. 29A is a perspective view of another embodiment of the present invention.

FIG. 29B is an exploded view of a dispenser that may be used with the present invention.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure or system. Further, the terms and phrases used herein are not intended to be limiting, but rather to provide an understandable description of the invention.

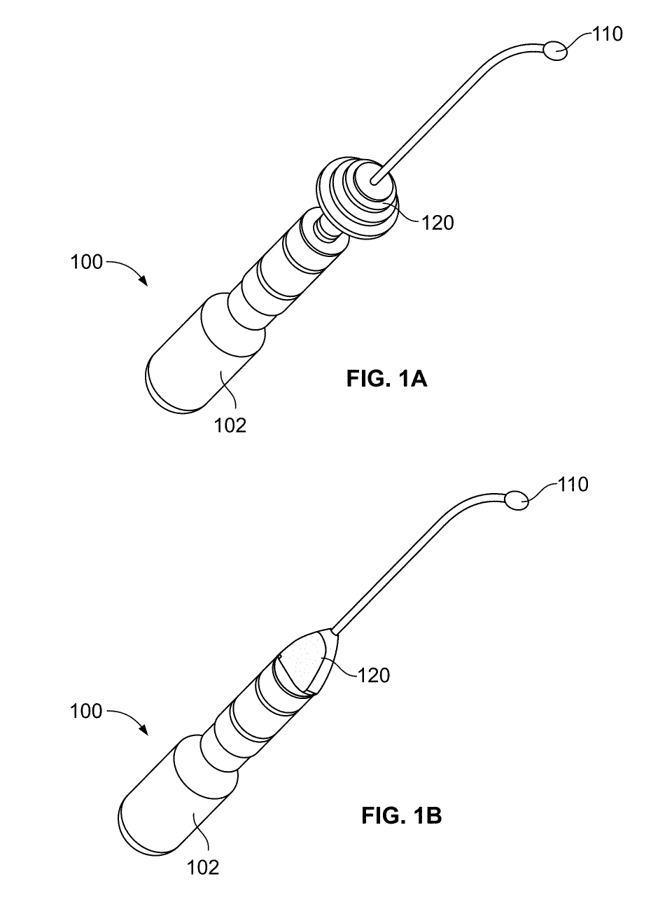

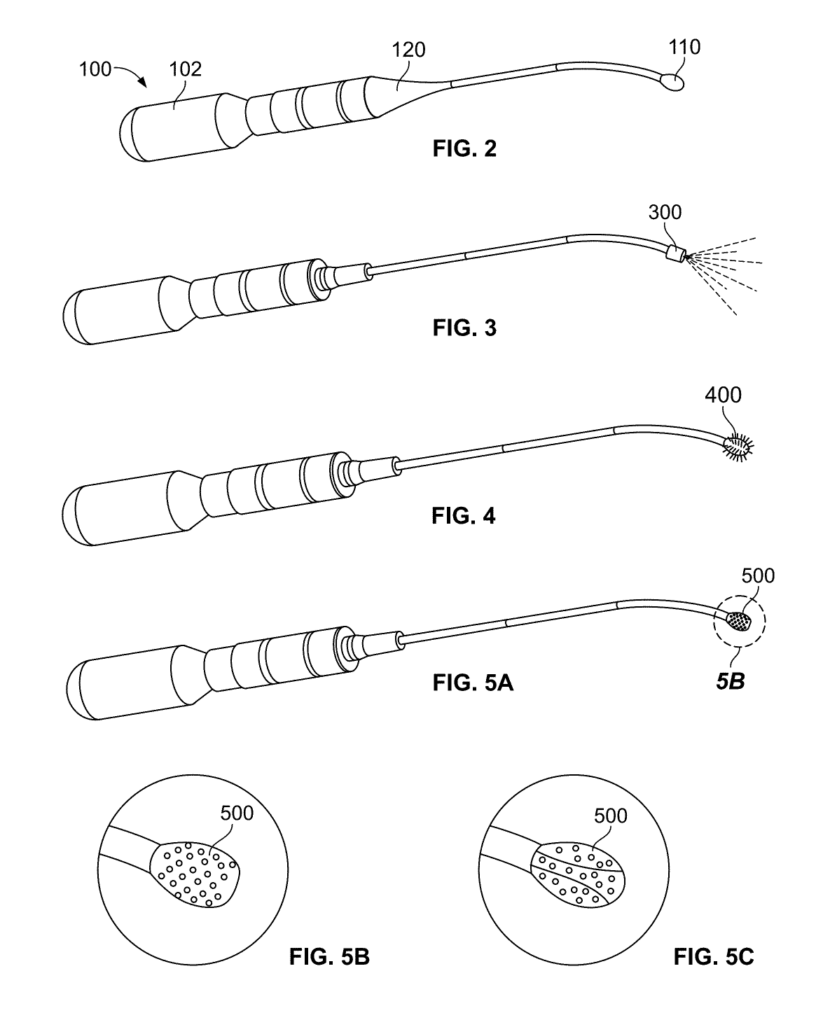

As shown in FIGS. 1-2, in one embodiment, the present invention includes a medical delivery device 100 having housing or base 102 and nozzle 104. The housing 102 is configured to contain a medicament. Nozzle 104 may be elongated and have a terminal end 110. Fitting 120 may also be provided with anchors and enhances the use of the device by assisting in the proper positioning of nozzle 104. As shown in FIG. 2, fitting 120 may be conical in shape and in other configurations as will be described below. Fitting 120 ensures the proper positioning of the nozzle 104

Terminal end 110 may be comprised of a variety of interchangeable components or tips as shown in FIGS. 3-7. For example, as shown in FIG. 3, the terminal end may be a spray head 300. Spray head 300 may be configured to have a spray pattern that can direct a liquid to a specific location or have multiple openings for a wider spray pattern.

As shown in FIG. 4, the terminal end may be a dispenser 400 that may have a plurality of filaments, tubes or hair-like tentacles to optimize delivery of the medication by increasing the surface area that can be contacted and by controlling the amount of medication that flows through the dispenser. Also, the length and number of filaments/tubes may be varied along the surface area to control the amount of medicament applied to a desired area.

As shown in FIGS. 5A-5C, the terminal end may be a dispenser 500 that may have a plurality of openings or perforations or be a porous or a semi-porous membrane. The openings and/or membrane optimize delivery of the medication by increasing the surface area that can be contacted and by controlling the amount of medication that flows through the dispenser. Also, the size and number of holes and/or porosity may be varied along the surface area to control the amount of medicament applied to a desired area.

FIG. 5C shows dispenser 500 is divided into three delivery areas. The divided dispenser 500 delivers the contents of individual lumens to separate areas.

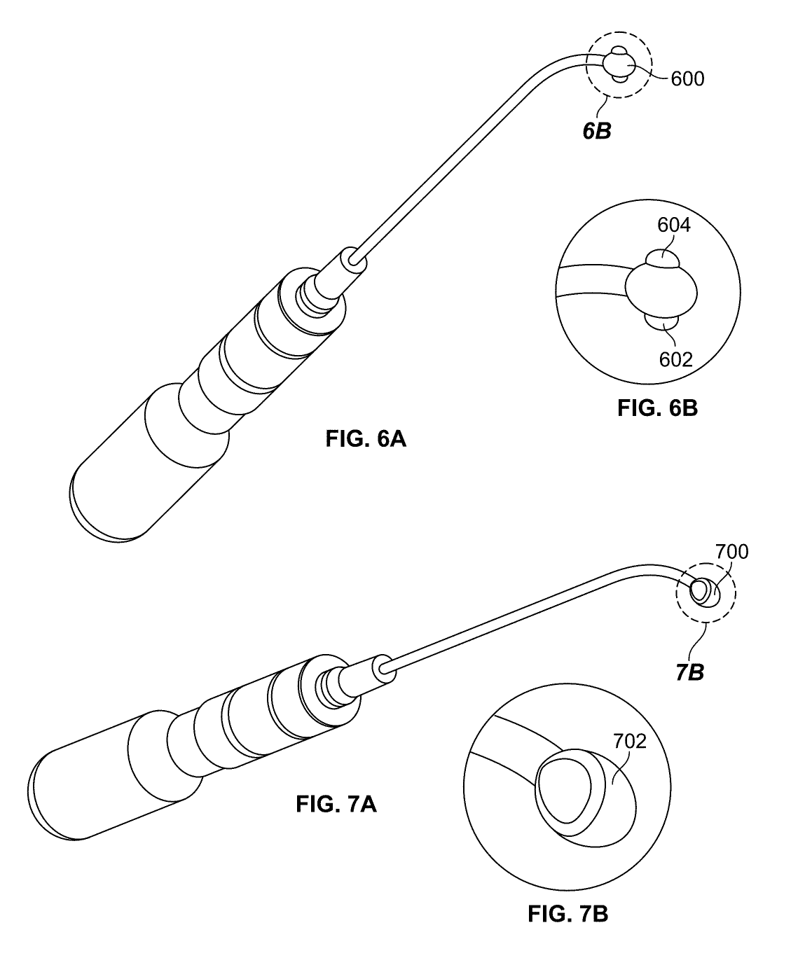

As shown in FIGS. 6A and 6B, the terminal end may be a dispenser 600 that may have a plurality of nodules or protrusions 602 and 604 that interface with a target area. The nodules may be permeable or configured in a manner similar to the embodiments described above such as in FIGS. 3-5. The nodules are designed to optimize delivery of the medication by increasing the surface area that can be contacted and by controlling the amount of medication that flows through the dispenser. Also, the size and number of nodules may be varied along the surface area to control the amount of medicament applied to a desired area.

As shown in FIGS. 7A and 7B, the terminal end may be a dispenser 700 that may have a shape that resembles a Q-tip or be asymmetric in nature. Also, dispenser 700 may have a shape that is one-half, one-quarter of a Q-tip or otherwise shape-optimized. A Q-tip with one surface flattened 702 is shown in FIG. 7B.

The various embodiments of dispensing tips mentioned above effectively improve drug delivery substantially. They also facilitate achieving topical mucosal tip delivery as well. The dispensing tip contacts with specific locus or loci, which is difficult for any disclosed prior art due to the varying and uneven surface anatomy and geography.

For example, current Q-tip device tip is suboptimal because of its shape, lack of maneuverability, lack of ability to alter or regulate local mucosal or tissue contact pressure or “site docking.” Suboptimal surface contact area/geometry poor regulation of rate or amount of medicament delivery, discharge of agent, compound or medication prior to reaching target, a large volume is required, causes unwanted nasopharyngeal delivery or loss of agent, aspiration of medication, swallowing of medication, trauma or abrasion to tissues during placement or removal, poor ease of placement, poor device anchoring or stability once delivery site contact accomplished. Suboptimal tensile properties allowing breakage, dislodgement, tissue embedding or penetration foreign body reaction or neural/CNS uptake, aspiration or inhalation of fiber/filament segments particles, drag or entanglement during placement or removal with altered tip geometry, affecting compound delivery to site and removal, tissue abrasion and fiber/filament breakage fragmentation,with tissue embedding, penetration, foreign body reaction, tissue uptake, aspiration, inhalation. Also, issues during manufacturing, uniformity, component assembly and packaging are significant.

The various embodiments of tip dispensers disclosed above may have the flattened or non drug delivery surface to rest against the nasal septum Static Expandable, balloon centered or off-centered, Deformable, Distensile via insert, screw mechanism, umbrella type mechanism, Directable, hydraulic, tumescent directed, cable, or spring directed insert, accordion elbow,or, bendable, or pliable connector allowing deflection in one or more planes at a given angle, rotational or screw directed drug delivery. The tip may be Pre soaked; with integral, proximal or distal reservoir., compound of one or more than one component, premixed, mixable, delivered at one locus, more than one locus, separate locus for each component. Single lumen, multi luminal Central location of swab delivery, florette delivery, Fiberoptic like array swab skeleton.

The various embodiments of tip dispensers disclosed above may be detachable or adhesable to delivery site Clipable, micro staple, barbed suture pushrod, balloon, gel, foam, adherent protein, polymer or patch.

The various embodiments of tip dispensers disclosed above may be integral to a unit delivery device but may be distinct stably attached module.

The various embodiments of tip dispensers disclosed above may be made of polymer, gel, gelatin, elastomer, resin, putty, fiber, filament, knit, weaved, braided, natural, cotton, silk, spider web, silk polyester, aminoacid, protein, collagen, Kevlar, wool, gut, leather, skin, collagen, cellulose, seaweed, or seaweed extract, algin, carbon fiber, composite, hollow fiber, tubular, non-friable, adsorbent absorbent, sponge like, polymer, plastic, or rubber soft pumice like with or without tubular/multi tubular.

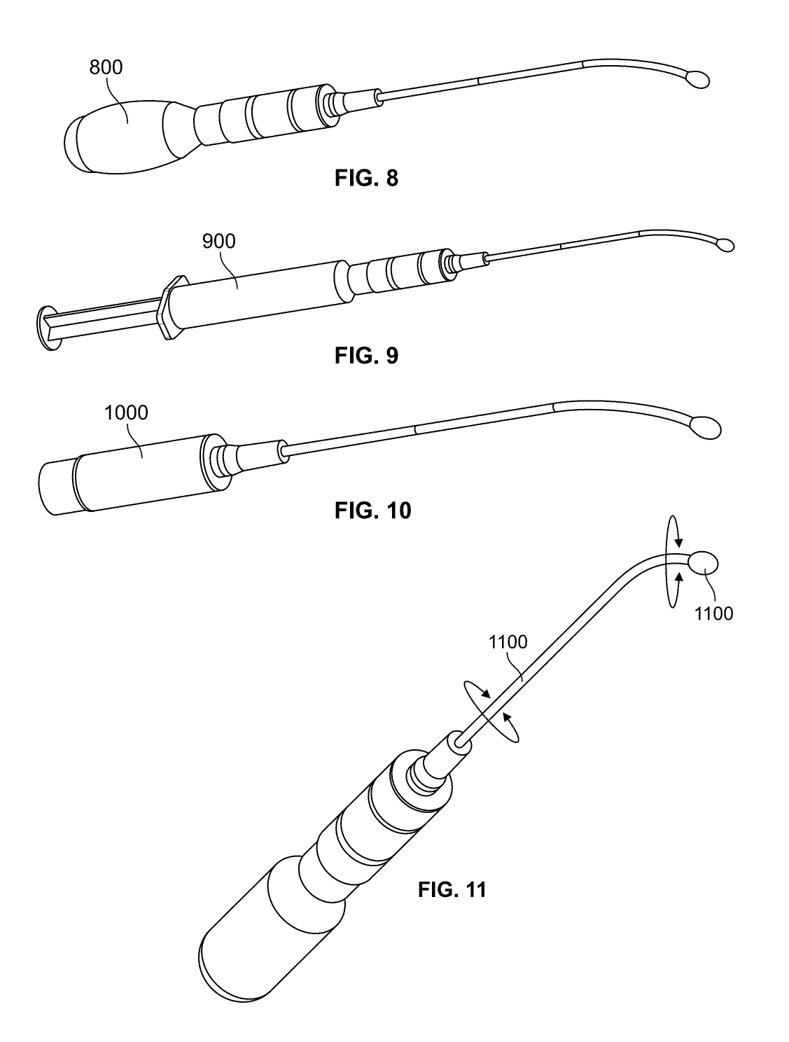

As shown in. FIGS. 8-10, a variety of drug delivery mechanisms may be used to propel a liquid through the device. As shown in FIG. 8, squeeze bulb 800 may be provided. As shown in FIG. 9, plunger 900 may be provided. As shown in FIG. 10, pressurized container 1000 may be provided.

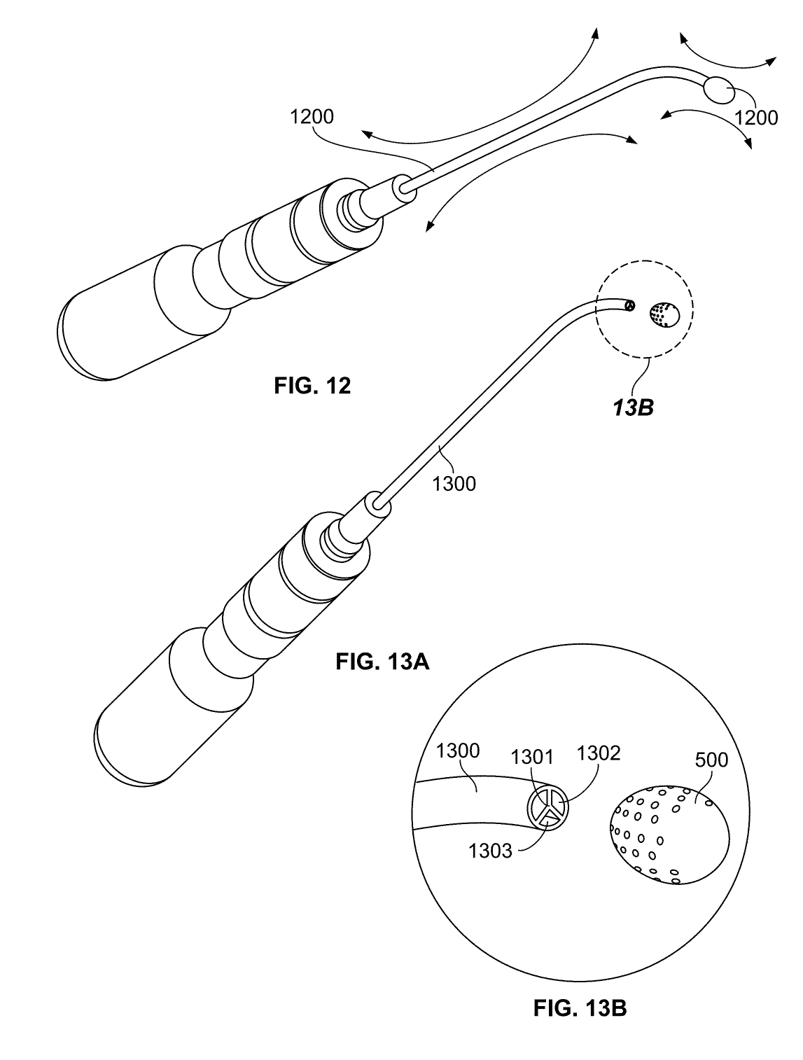

As shown in FIG. 11, nozzle 1100 may be rotatable as well as extendable in a variety of directions. As shown in FIG. 12, shaft 1200 may be bendable to optimize positioning or the pressure exerted by a terminal end. The shaft may also be bendable in arc-like and other translations as well as extendable in a vertical direction. These designs will aid in delivering a medicament to a desired area. Also, as described above, for all embodiments of the present invention, the various terminal end configurations described may be used with these designs since they are designed to be interchangeable.

As shown in FIGS. 13A-13B, the nozzle configurations shown above may be in the form of an elongated tube 1300 that may be further configured to have a plurality of lumens 1301-1303 as shown in FIG. 13B. The lumens may be uniform in cross-section or have different volumes to control the amount of medicament applied to a specific area. The lumens may also include filaments or other permeable materials as well as baffles and other types of inclusions therein to assist in directing the flow of a medicament to a predetermined area.

The bending mechanisms used to bend the bendable components in the present invention may be straw elbow, preformed geometry, air, gel or hydrostatic, cable directed, catheter or preformed insertion or deformable, or extension or pre tension insert. The advancing or extending or elongating or retracting mechanisms used in the present invention may include: Nasal anatomic or another plug, soft, comfortable circumferentially around tube etc. The bending and or elongation may be fixed, movable; slide, friction, screw mechanism, click in place mechanism, combination.

The present invention uses luminal geometry or asymmetry to deflect or direct insert, tube, or agent in part or entirety.

In other embodiments, one or more lumens may be used to change the configuration of tubes 1300 by including therein wires or some other mechanical stiffening materials. Pressurized hydraulics may also be used as well as electrical mechanical substances.

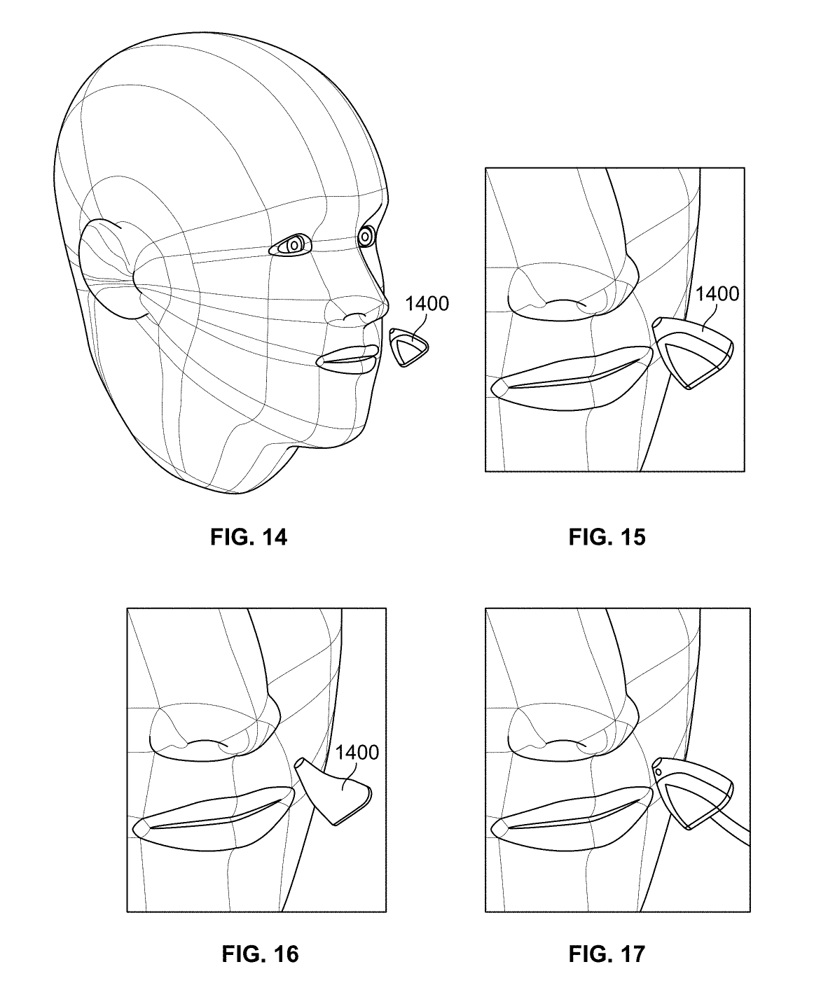

As shown in FIGS. 1-2, a fitting 120 may be provided. The purpose of the fitting is to function as an anchor against a subject to ease in the insertion of a nozzle. In one embodiment, shown in FIG. 2, the fitting 120 has an elongated cone shape to ensure proper positioning. The nozzle can be a separate piece or be integrated into the delivery tube. In other embodiments, as shown in FIGS. 14-17, fitting or anchor 1400 is provided which is adapted to fit securely into the naris, performing as an anchoring nares nosepiece. The purpose of this anchor is to be fitted securely into the naris of humans or animals to allow the delivery of agents to various targeted areas of the nasal cavity to provide a platform to guide ancillary devices to anchor and guide for delivery systems.

As shown in FIG. 15, the device 1400 is generically shaped in the form of the naris. It can retain this shape during use. In another embodiment, the nosepiece 1400 can be constructed of a malleable material to conform with the specific anatomical situation.

FIG. 16 shows an apertures 1604 on the nosepiece 1400. Agent may be delivered via one or more apertures which can be positioned in various positions and orientations to optimal targeting.

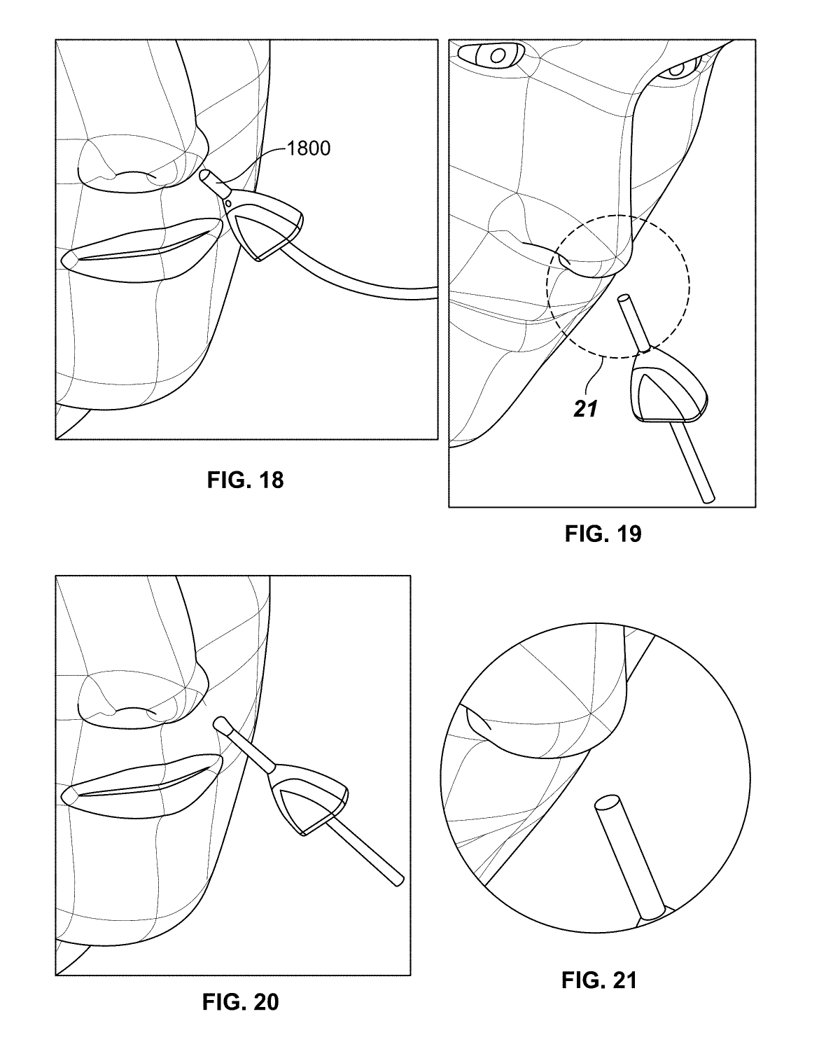

As shown in FIGS. 18-21, the anchor may be adapted to receive a tube as shown. Also, the anchor may be adapted to receive the above-described embodiments such as those shown in FIGS. 1-13.

In other embodiments, as shown in FIG. 18, tube 1800 may have a distal end that pops up after insertion and can be driven by hydraulic, pressurized or other means.

Also, the anchor may have a terminal end that may be in one of the configurations described above. This terminal ends may be interchangeable as well.

In other aspects, the present invention provides a terminal end or tip design that is configured to provide maximum medicament delivery while minimizing any inadvertent loss into unintended areas. In one embodiment, the tip may be a textile. The textile may be a 3D structure or a specific anatomical design or impart key performance criteria, such as permeability in a particular direction or area. Suitable coating may be used that have the following characteristics: impermeable sealant, hydrophilic or hydrophobic coatings, adhesive layers, radiopaque markers, placement indicators etc . . . . In other embodiments, the textile may be condensed which reinforces the strength profile of the construct, offers a smooth feel, a lower coefficient of friction, as well as a lower profile and increased surface area for drug delivery.

Other components may be integrated into the textile to increase functionality such as bonding, permeability, impermeability as well as the ability to work with other medical instruments. The textile may also be looped, sliced and have other enhancements such as braiding to increase the controlled delivery of a medicament.

In other aspects, the present invention provides a tip made of hybrid fabrics that include more than one type of structural fiber in its construction. Hybrid fabrics allow the fibers to be constructed into one or more layers of fabric. In a woven hybrid fabric, it is possible to have one fiber running in the weft (crosswise) direction and the second fiber running in the warp (longitudinal) direction. It is also possible to use a combination of different fiber types in each warp and weft direction to control the delivery of a medicament.

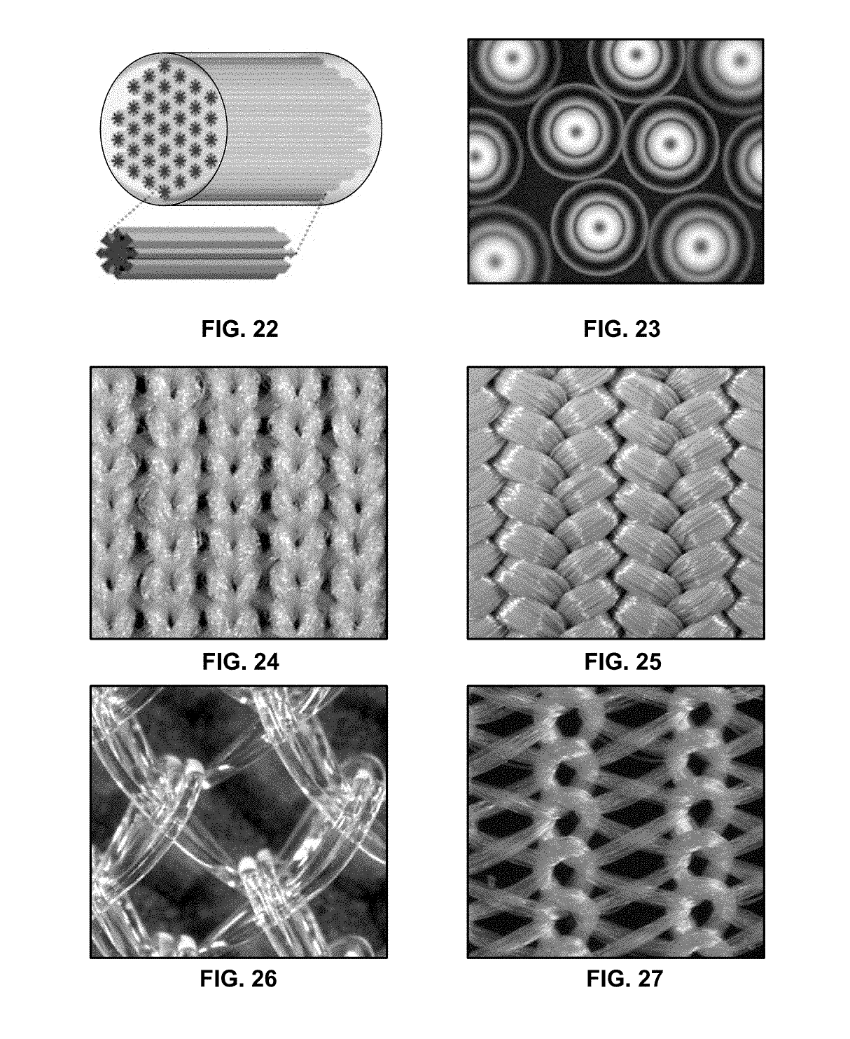

In other aspects, as shown in FIGS. 22-23, the present invention provides a tip made from nonwoven fabrics which is broadly defined as a sheet or web structure bonded together by entangling medical fibers or filaments (and by perforating films) mechanically, thermally or chemically. They are flat, porous sheets that are made directly from separate fibers or from molten plastic or plastic film.

The fibers in a non-woven may be oriented in one direction or randomly throughout the fabric. Multiple layers can be combined to achieve desired strength, elongation and other mechanical properties. Porosity can be controlled by varying fiber diameter, fiber density, fiber orientation and additional mechanical processing.

In other aspects, the present invention provides a tip that employs a braided design. A braid is a complex structure or pattern formed by intertwining three or more strands of flexible material such as textile fibers or wire. A braid is usually long and narrow, with each component strand functionally equivalent in zigzagging forward through the overlapping mass of the others that may be arranged to control the application of a medicament.

In other aspects, the present invention provides a tip that employs warp knitting which is a method of constructing fabric by interlocking a series of loops of one or more yarns. Warp knitting is a type of knitting in which the yarns generally run lengthwise in the fabric and may be arranged to control the application of a medicament.

In other aspects, the present invention provides a tip that employs weaving as shown in FIGS. 24-27. A woven fabric is comprised of warp yarns in the longitudinal direction and weft yarns in the crosswise direction. The warp and weft yarns may be the same fiber and same size or they may be different in fiber type and size to control the application of a medicament.

In other aspects, the present invention provides a tip that employs a high-definition microextrusion (HDME). This technique uses nano- and micron-sized fibril components to form precise, unique structures within a fiber. The level of detail and definition using the HDME process enables intricate polymer domains within a fiber.

The HDME process makes it possible to use up to four polymers during the manufacturing process to produce a wide variety of customized fibers. Another unique capability of the HDME process is the production of spun fibers with diameters of 300 nm using an islands-in-the-sea technique coupled with dissolvable and nondissolvable materials. This technique also enables the use of a small number of strong permanent fibers coupled with dissolvable fiber material to produce a lower profile final fiber.

The fiber may be Nonbiocompatible or biocompatible; Soft, Pliable yet resistant to abrasion, degradation, fatigue, fragmentation, fracture, tearing, adverse deformation, Nonresorbable.

Some Examples of biocompatible polymers are Polyethylene, UHMWPE ultra high weight molecular Polyethylene Polypropylene, Polyethylene Terephtalate (PET), Polyurethane (PU), Polymethyl Methacrylate (PMMA) Polyetheretherketone (PEEK), Resorbable, Polylactic Acid (PLA), Polyglycolic Acid (PGA) Polylactide/Glycolide Copolymers (PLGA) Polycaprolactone (PCL), Poly-4-hydroxybutyrate (P4HB), Chitosan Alginates, Polyacrylonytrile, Toluene 3, 4 diisocynate & Polyethylene glycol Polyamide, Polyacrylate or poly acrylic acid, Polyterafluoroethylene, Other Polymers Used in Medical Textiles, Polydioxanone, Polyethylene Polyglycolic Acid, Polylactic Acid, Polyactide, Polyglycoide, Poly (N-isopropyl acrylamide).

The essential characteristics for fibres to be used in spunlaced technology are as follows (Adanur, 1995; Madhavamoorthy and Shetty, 2005).

1. Modulus: Fibers with low bending modulus requires less entangling energy than those with high bending modulus.

2. Fineness: For a given polymer type, larger diameter fibers are more difficult to entangle than smaller diameter fibers because of their greater bending rigidity. For PET, 1.25 to 1.5 deniers appear to be optimum.

3. Cross section: For a given polymer type and fiber denier, a triangularly shaped fiber will have 1.4 times the bending stiffness of a round fiber. An extremely flat, oval or elliptical shaped fiber could have only 0.1 times the bending stiffness of a round fiber.

4. Length: Shorter fibers are more mobile and produce more entanglement points than longer fibers. Fabric strength, however, is proportional to fiber length; therefore, fiber length must be selected to give the best balance between the number of entanglement points and fabric strength.

5. Fiber wetability: Hydrophilic fibers entangle more easily than hydrophobic fibers because of the higher drag forces.

The present invention is nontoxic, nonreactive, nonallergenic, and sterilizeable.

The present invention may be used Site Specific Directed, or Directed NonSite Specific, or Nondirected Delivery. The use case of the present invention May be Nonselective, Selective, Combined, systemic,local, topical as noted above may be CNS, neuronal, sinus. Nasopharyngeal, esophageal, tracheal, eustacian, Aural, oral, dental etc.

Some or all of the detachable components in this present invention may be attached snap on, screw on, radial placed multiple latches, circular snap, friction fit or with stippling, adhesive, vacuum.

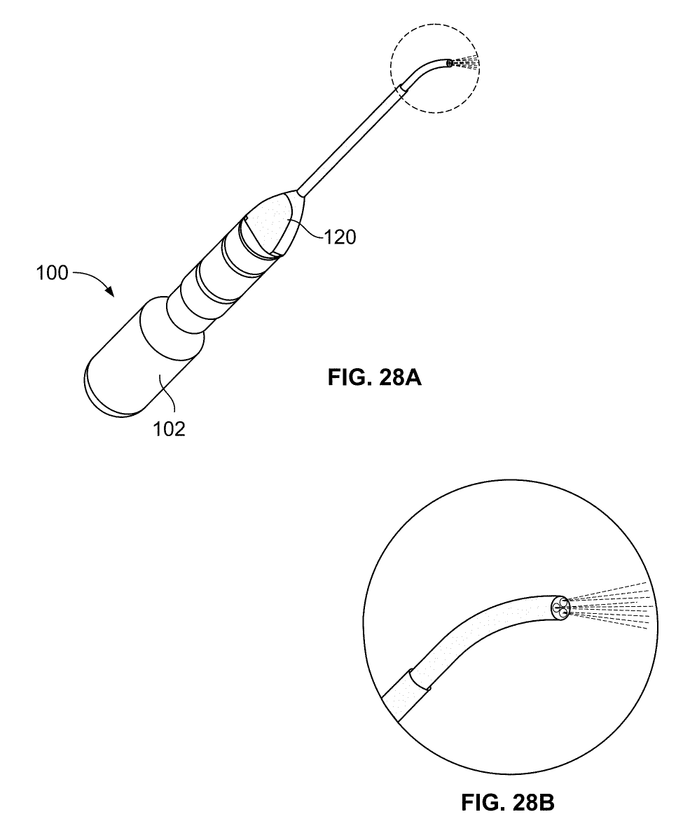

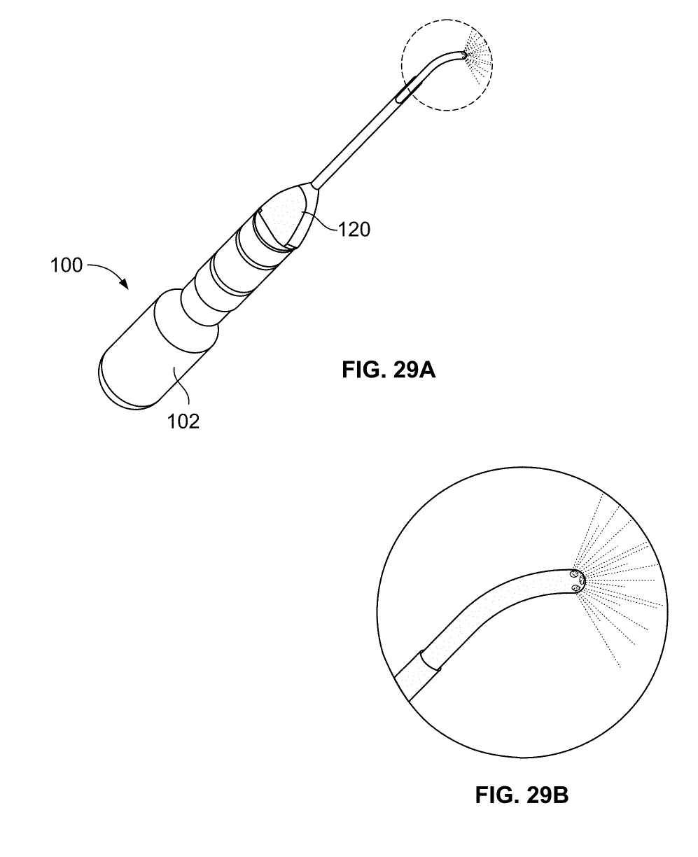

As shown in FIGS. 28 and 29, on other embodiments, the present invention provides a nozzle that provides a mist or spray. The nozzle may have a varied or variable angle for SPG or other locus, ie: cribriform plate/olfactory nerve or multiple dorsonasal loci for Narcan or other narcotic antagonist going to Brain or CSF for better reversal of respiratory or cardiac depression for improved salvage or lifesaving efficacy in overdoses.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above-described embodiments, methods, and examples, but by all embodiments and methods within the scope and spirit of the disclosure. The disclosure also includes the materials attached hereto.

What is claimed is:

1. A drug delivery system comprising:
a housing configured to contain a medicament;
a drug delivery mechanism to expel medicament from a nozzle;
a fitting adapted to properly connect said nozzle and said housing via said drug delivery mechanism; and
said nozzle having a tip to control the application of a medicament, said tip has at least one opening.

2. The device of claim 1 wherein the tip is positionable.

3. The device of claim 1 wherein the tip is adapted to optimize said medicament delivery contacting surface and flow.

4. The device of claim 3 wherein the tip is a spray head.

5. The device of claim 3 wherein the tip is selected from the group consisting of a plurality of filaments, tubes, and hair like tentacles.

6. The device of claim 3 wherein the tip is divided to deliver said medicament to separate areas.

7. The device of claim 3 wherein the tip has at least one nodules.

8. The device of claim 3 wherein the tip has at least one flattened surface.

9. The device of claim 1 wherein said drug delivery mechanism is selected from the group consisting of at least one squeeze bulb, plunger and pressurized container.

10. The device of claim 1 wherein the nozzle is positionable.

11. The device of claim 1 wherein the nozzle is extendable and retractable.

12. The device of claim 1 wherein the nozzle has a plurality of lumens.

13. The device of claim 12 wherein the lumens is configured to change the shape of said nozzle.

14. The device of claim 1 further including an anchor to assist in positioning the device.

15. The device of claim 13 wherein the anchor is adapted to receive a distal end.

16. The device of claim 1 wherein the tip is a textile.

17. The device of claim 16 wherein the textile tip is treated with at least one method selected from the group consisting of bonding, looping, slicing, braiding, woven, nonwoven, non-woven in one direction, non-woven randomly throughout the fabric, combining multiple layers, warp knitting, weaving, and HDME.

18. A method of administering a medicament to a user comprising:
providing a drug delivery device, said drug delivery device comprising a housing configured to contain a medicament;
a drug delivery mechanism to expel medicament from a nozzle;
a fitting adapted to properly connect said nozzle and said housing via said drug delivery mechanism; and
said nozzle having a tip to control the application of a medicament, said tip has at least one opening;
operating said drug delivery mechanism;
expelling said medicament from said nozzle; and
dispensing said medicament to at least one targeted area from said tip.

19. A drug delivery system comprising: a housing configured to contain a medicament;
a drug delivery mechanism to expel medicament from a nozzle having a plurality of lumens;
a fitting adapted to properly connect said nozzle and said housing via said drug delivery mechanism; and
said nozzle having a tip to control the application of a medicament, said tip has at least one opening.

20. The device of claim 19 wherein the tip is positionable.
